(19) United States
(12) Patent Application Publication
(10) Pub. No.: US 2025/0107607 A1
(43) Pub. Date: Apr. 3, 2025

(54) Camera Sensor

(71) Applicant: L'Oreal, Paris (FR)

(72) Inventors: Gregoire Charraud, Jersey City, NJ (US); Rafael Feliciano, Jersey City, NJ (US)

(73) Assignee: L'Oreal, Paris (FR)

(21) Appl. No.: 18/812,180

(22) Filed: Aug. 22, 2024

Related U.S. Application Data
(60) Provisional application No. 63/586,205, filed on Sep. 28, 2023.

(30) Foreign Application Priority Data
Nov. 27, 2023 (FR) 2313066

(51) Int. Cl. A45D 20/12; A45D 34/04; A45D 44/00; A61K 8/04; A61Q 5/06; G06T 7/60; G06T 7/90

(52) U.S. Cl. CPC A45D 20/12; A45D 34/04; A61K 8/046; A61Q 5/065; G06T 7/60; G06T 7/90; A45D 2044/007; A45D 2200/155; G06T 2207/30196

(57) ABSTRACT

A camera sensor attachment for a hair dryer for facilitating application of an aerosolized cosmetic composition, such as a hair dye, to hair of an individual. A hair dryer that includes a camera sensor attachment. A method of using the camera sensor attachment and/or a hair dryer that includes the camera sensor attachment attached thereto for applying an aerosolized cosmetic composition. Computational device-implemented variations of devices and methods.

### CROSS-REFERENCE(S) TO RELATED APPLICATION(S)

This U.S. Nonprovisional patent application claims the benefit of U.S. Provisional Patent Application No. 63/586,205 filed on Sep. 28, 2023, and French Patent Application No. FR 2313066 filed on Nov. 27, 2023; the contents of which are incorporated herein in their entirety for all purposes.

### SUMMARY

In an aspect, the disclosure provides an attachment for a hair dryer, the attachment comprising a camera sensor operably connected to circuitry; wherein the circuitry is configured to image, with the camera sensor, a portion of a body of an individual for a diagnosis of the portion of the body and an application of an aerosolized cosmetic composition to the portion of the body based at least in part on the diagnosis.

In embodiments, the attachment is configured for physical attachment to the hair dryer.

In embodiments, the attachment is an after-market addition to the hair dryer.

In embodiments, the aerosolized cosmetic composition is for application to hair of an individual.

In embodiments, the aerosolized cosmetic composition includes a hair dye that is curable with hot air of the hair dryer, wherein the attachment includes an ion generator for generation of ions in the hair dye prior to application to hair of the individual.

In embodiments, the diagnosis corresponds to a natural strand color, a strand density, a strand thickness, a strand length, one or more artificial strand colors, or any combination thereof.

In embodiments, the circuitry is configured to compute a hair dryer blower level, a hair dryer blower temperature, and/or an applicator spray level based at least in part on the diagnosis.

In embodiments, the circuitry is configured to: activate an applicator of the attachment for application of an aerosolized cosmetic composition to the portion of the body of the individual; and activate a blower of the hair dryer; wherein the hair dryer blows air based at least in part on the hair dryer blower level, the hair dryer blower temperature, and/or the applicator spray level.

In an aspect, the disclosure provides a hair dryer system that comprises the attachment, wherein the attachment is attached or is attachable a hair dryer of the hair dryer system.

In an aspect, the disclosure provides a method for applying an aerosolized cosmetic composition to a portion of a body of an individual, the method comprising: imaging, with a camera sensor of an attachment for a hair dryer, the portion of the body to produce an image, wherein the attachment is attachable to the hair dryer; computing, with circuitry, a diagnosis of the portion of the body of the individual based at least in part on the image; computing, with circuitry, a hair dryer blower level, a hair dryer blower temperature, and/or an applicator spray level based at least in part on the diagnosis; applying, with an applicator, the aerosolized cosmetic composition to the portion of the body of the individual, and activating a blower of the hair dryer; wherein the hair dryer blows air based at least in part on the hair dryer blower level, the hair dryer blower temperature, and/or the applicator spray level.

In embodiments, the attachment is attached to the hair dryer.

In embodiments, a diagnosis area of the camera sensor, a spray of the applicator of the attachment, and a portion of the hot air blown by the hair dryer are coincident with the portion of the body.

In embodiments, the aerosolized cosmetic composition is for application to hair of the individual.

In embodiments, the aerosolized cosmetic composition includes a hair dye that is curable with hot air of the hair dryer.

In an aspect, the disclosure provides a computational device that comprises circuitry configured to perform all or part of a method of the disclosure.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1 shows a diagram of an example attachment for a hair dryer that includes a camera sensor, operable with the hair dryer for image-based diagnosis of a portion of the hair, as well as applying and curing a cosmetic composition to the hair, according to aspects of the disclosure.

FIG. 2 shows a side view of an example attachment for a hair dryer that includes a camera sensor, attached to the hair dryer for image-based diagnosis of a portion of the hair, as well as applying and curing a cosmetic composition to the hair, according to aspects of the disclosure.

FIG. 3 shows a side view of the example attachment for the hair dryer that includes the camera sensor, attached to the hair dryer, applying and curing a cosmetic composition to the hair, according to aspects of the disclosure.

FIG. 4 shows a flowchart of an example method for applying a cosmetic composition to an individual's hair based at least in part on an image-based analysis or diagnosis of the individual's hair, according to aspects of the disclosure.

TABLE 1
Drawings elements and descriptions.

| Reference | Description |
|---|---|
| 1 | Hair dryer system |
| 2 | Aerosol attachment device |
| 3 | Hair dryer |
| 4a | Rearward securement (aerosol attachment device) |
| 4b | Forward securement (aerosol attachment device) |
| 5a | Rearward securement (Hair dryer) |
| 5b | Forward securement (Hair dryer) |
| 6 | Securement rail |
| 9 | Individual's hair |
| 10 | Method for applying an aerosolized cosmetic |
| | composition to an individual's hair with image- |
| | based diagnosis |
| 11 | Preparing an individual's hair for application of a |
| | cosmetic composition |
| 12 | Imaging the hair with a camera sensor attachment |
| | and applying the cosmetic composition to the |
| | individual's hair with an applicator based on |
| | analysis of an image of the hair |
| 13 | Curing the cosmetic composition with hot air of the |
| | hair dryer |
| 14a | Nozzle head |
| 14b | Nozzle aperture |
| 15 | Camera sensor attachment |

The foregoing aspects and many of the attendant advantages of this disclosure will become more readily appreciated as the same become better understood by reference to the following detailed description, when taken in conjunction with the accompanying drawings.

### DETAILED DESCRIPTION

Existing devices and methods for hair coloring typically utilize an oxidative chemical reaction that can be corrosive to tissues and is overly time consuming. With these approaches, typically, two compounds are mixed to form a hair dye composition, and the hair dye composition is then meticulously applied to the hair. These approaches take time but can be able to produce colored hair that lasts for three or more weeks. There is a need for devices and methods for applying hair dye compositions onto the hair and curing the hair dye compositions to form satisfactory lasting of the colored hair. The present disclosure addresses this and other long-felt and unmet needs in the art.

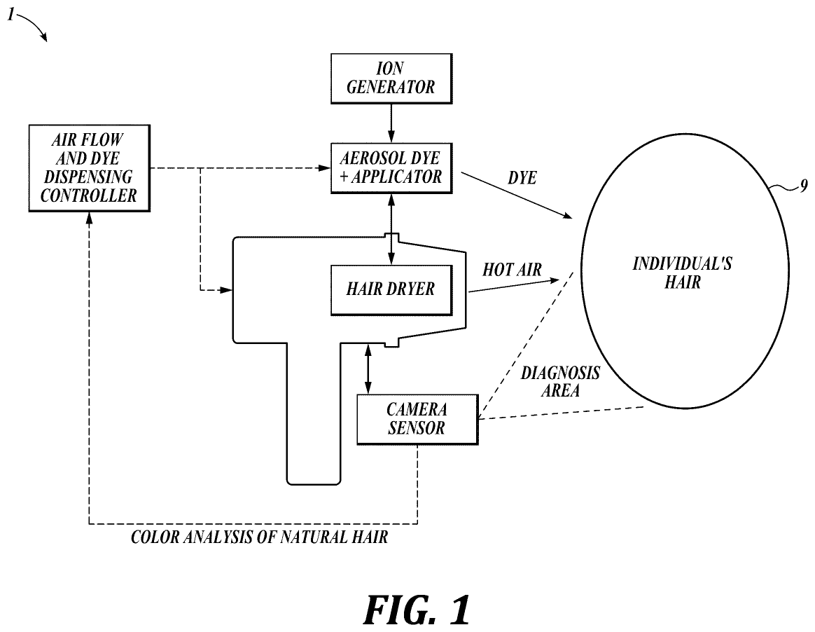

FIG. 1 shows a diagram of a hair dryer system 1 with a camera sensor attachment attached thereto, as can be used to apply an aerosolized cosmetic composition on an individual's hair 9 and cure the aerosolized cosmetic composition with the hair dryer, according to aspects of the disclosure. An example attachment for a hair dryer comprises a camera sensor that is configured to image an individual's hair, within a diagnosis area, for analysis or diagnosis of the hair for management of application of a cosmetic composition, such as a hair dye, to the individual's hair. The applicator can spray aerosolized hair dye within an area that is adjacent to or overlapping with an output of hot air of the hair dryer when attached to the hair dryer, such that the hot air emitted by the hair dryer is at least partially coincident with the spray of the attachment and a diagnosis area of the camera sensor. In embodiments, the hair can be analyzed by circuitry of a computational device (e.g., a “smart hair dryer” computational device) for a diagnosis to determine, for example, one or more desired settings of the hair dryer system 1, a degree of coverage of hair within the diagnosis area with the cosmetic composition, one or more options for the hair dye composition, or the like, and the composition can be applied and cured with the hot air for improved applying and curing of the cosmetic composition.

Circuitry of the hair dryer system 1 can be configured as an air flow and dye dispensing controller, as shown at FIG. 1, which can include circuitry configured to analyze imagery of the individual's hair 9 captured by the camera sensor. In embodiments, the diagnosis corresponds to a natural strand color, a strand density, a strand thickness, a strand length, one or more artificial strand colors, or any combination thereof. For example, a natural hair color can be computed and used as the basis for computation of one or more suggested artificial hair colors for an individual to consider or select. As another example, the individual's hair strand density, thickness, length, or the like, can be computed and used as the basis for computation of one or more suggested cosmetic compositions for the individual to consider or select for application to their hair, such as shampoos, conditioners, thickening agents, hair dye compositions, or the like.

In embodiments, a hair dryer blower level, a hair dryer blower temperature, and/or an applicator spray level can be computed by circuitry of the system, based at least in part on a diagnosis computed by the circuitry. For example, the hair dryer blower level and/or the hair dryer blower temperature can be computed to be desirably higher if the hair is detected to be wetter, so as to effectively dry the hair and cure the composition, and/or can be computed to be desirably lower if the hair is detected to be dryer, so as to avoid overheating the scalp. As another example, the applicator spray level can be computed to be desirably higher if a portion of the hair is detected to be lacking in the cosmetic composition thereon, and/or can be computed to be desirably lower if a portion of the hair is detected to be sufficiently covered with the cosmetic composition thereon. Furthermore, in embodiments, the diagnosis can be computed by the circuitry continuously during use of the hair dryer system, and one or more parameters, e.g., the hair dryer blower level, the hair dryer blower temperature, and/or the applicator spray level, can be continuously computed and updated in response to changes during the application process. In this manner, the hair dryer system 1 can automatically adjust the parameters for a more consistent application of the cosmetic composition to the individual's hair 9.

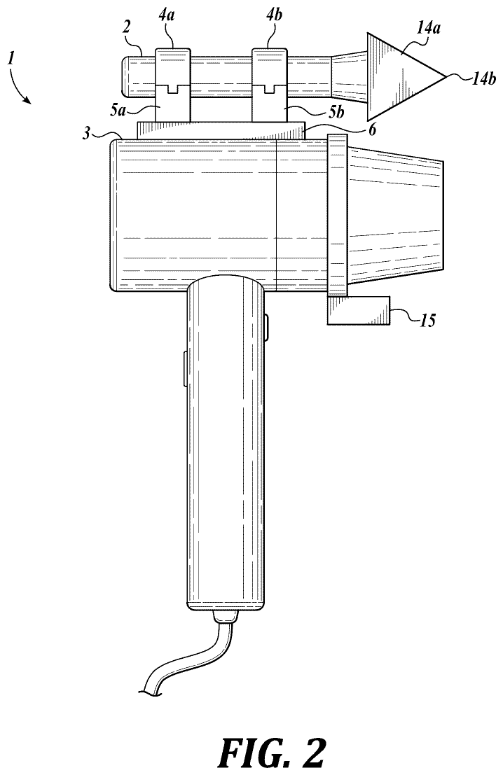

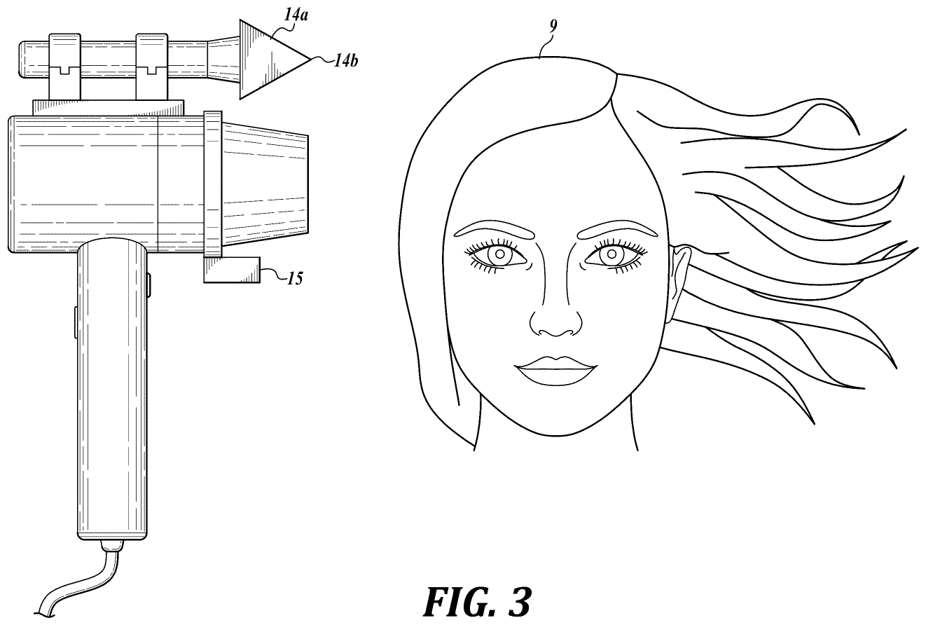

As shown at FIGS. 2 and 3, an example hair dryer system 1 for aerosolized application, image-based analysis, and heat curing of a cosmetic composition comprises an aerosol attachment device 2 affixed to a hair dryer 3. In embodiments, an aerosol attachment device 2 can be provided as an after-market device for attachment to a commercially available hair dryer 3, such as by way of rearward and forward securements 4a and 4b that are securable around the aerosol attachment device 2, and rearward and forward securements 5a and 5b that are securable around the hair dryer 3. Securements 4a and 4b can be connected to securements 5a and 5b to secure the aerosol attachment device 2 to the hair dryer 3.

In embodiments, securements 4a and 5a, and 4b and 5b, can be connected together with any suitable fastener, including but not necessarily limited to screws and threaded holes, hook and loop fasteners, complementary magnets, snap connectors, buckles, buttons, zippers, straps, and the like. In embodiments, positions or configurations of securements 4a and 5a, and 4b and 5b are adjustable, such that an angle of the aerosol attachment device 2 relative to the hair dryer 3 is adjustable, for example, for adjustment of alignment of a path of a dye composition sprayed from the aerosol attachment device 2 with a path of blown air of the hair dryer 3. Since different hair dryers 3 can be shaped differently, resulting in different angles between the aerosol path and the path of blown air, adjustment of the angle between the aerosol attachment device 2 and the hair dryer 3 can enable compensation for these differences and help the aerosol path be at least partially coincident with a point of impact of the blown air with hair of an individual during use of the system.

In various aspects, an attachment 15 for the hair dryer 3 includes a camera sensor that is operably connected to circuitry. The circuitry is configured to image, with the camera sensor of the attachment 15, a portion of a body of an individual for a diagnosis of the portion of the body and an application of an aerosolized cosmetic composition to the portion of the body based at least in part on the diagnosis. In embodiments, the attachment 15 is configured for physical attachment to the hair dryer 3, and can be connected to the hair dryer 3 with any suitable fastener, including but not necessarily limited to screws and threaded holes, hook and loop fasteners, complementary magnets, snap connectors, buckles, buttons, zippers, straps, and the like.

The aerosol attachment device 2 can comprise a dye composition and an ion generator configured to aerosolize the dye composition for being sprayed from an applicator of the aerosol attachment device. An applicator can comprise a nozzle head 14a and a nozzle aperture 14b; the nozzle head 14a can channel the dye composition into a smaller volume for being sprayed from the nozzle aperture 14b.

In the shown embodiment, the camera sensor attachment 15 is separate and distinct from the aerosol attachment device 2, for a degree of modularity of the hair dryer system 1. For example, in embodiments, the hair dryer 3 can include the attachment 15 thereon, for use without the aerosol attachment device 2. In this configuration, the camera sensor can analyze the individual's hair for modulation of parameters of the hair dryer 3, and the cosmetic composition can be applied to the individual's hair through other means, e.g., manually or with a separate device. In other embodiments, the camera sensor attachment 15 is an additional element of the aerosol attachment device 2, and can be mounted or secured to the hair dryer 3 along with the aerosol attachment device 2 as a unit.

In embodiments, the hair dryer 3 can be provided from a factory, or can be equipped as an after-market addition, with a securement rail 6. The securement rail 6 can be comprised of one or more ridges along a length thereof for securement of securements 4a and 4b, and 5a and 5b, thereto at any position along the length of the securement rail 6. In example embodiments and for purposes of illustration, the securement rail 6, and the securements 4a and 4b, and 5a and 5b, can be considered to function in a manner that corresponds with that of a firearm optics rail with optics attachments, or the like. However, in embodiments, the attachment is integrated or integral with the hair dryer, such that the system is configured as a unit.

As shown at FIG. 3, a hair dryer system can be used to cure and dry a cosmetic composition applied to an individual's hair. As shown, a hair dryer system can include the aerosol attachment device attached to the hair dryer, which includes a nozzle head 14a and a nozzle aperture 14b, as well as an attachment 15 that includes a camera sensor. During use, the aerosol attachment device can emit an aerosol spray path containing aerosolized dye composition, and the hair dryer can emit a blown air path. The aerosol spray path can be at least partially coincident with the blown air path at a portion of the individual's hair 9, or can contact the individual's hair at a position near or adjacent to a point of contact of the blown air path with the individual's hair, according to embodiments. A heat curable cosmetic composition (e.g., hair dye composition) can be cured by the blown air path, and the individual's hair dried of excess water or other liquid by the blown air path, simultaneously. In this manner, the time for curing and drying is reduced.

To facilitate this process, the camera sensor of the attachment 15 can capture images of the individual's hair and can transmit the images to circuitry configured to continuously analyze the individual's hair 9 and compute a diagnosis that can form the basis for desired parameters for operation of the hair dryer, the applicator, or both, for example. Accordingly, in embodiments, an attachment device for a hair dryer comprises an aerosol attachment device that is configured to cure a cosmetic composition, such as a hair dye, applied to an individual's hair based at least in part on the diagnosis. The aerosol spray path of the attachment can spray an area that is at, near, or adjacent to an output of the hair dryer when attached to the hair dryer, such that warm or hot air emitted by the hair dryer is at least partially coincident with the composition sprayed from the attachment, based at least in part on the diagnosis. In embodiments, the cosmetic composition can be simultaneously applied and cured for improved application of the cosmetic composition, based at least in part on the diagnosis.

In embodiments, the cosmetic composition is configured for application to the hair of an individual, such as the hair on the head. The cosmetic composition can be, or can include, a hair dye that is curable with hot air of the hair dryer. The hair dye can be for introduction of a hair color to the individual's hair, for example.

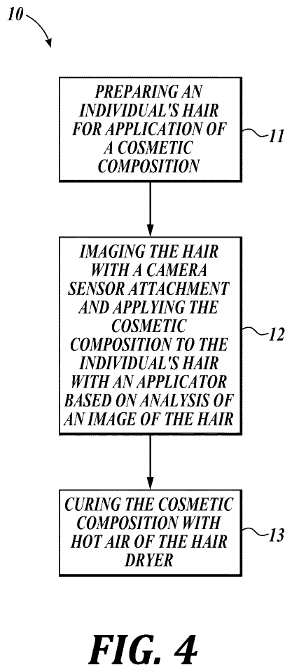

FIG. 4 shows a flow chart of an example method for applying an aerosolized cosmetic composition to an individual's hair with an example hair dryer system that includes a camera sensor. A method 10 can include, at step 11, preparing an individual's hair for application of a cosmetic composition; at step 12, imaging the hair with a camera sensor attachment and applying the cosmetic composition to the individual's hair with an applicator based on analysis of an image of the hair; and at step 13, curing the cosmetic composition with hot air of the hair dryer. In embodiments, the hot air contacts the cosmetic composition applied to the hair according to hair dryer parameters computed by circuitry. In embodiments, the attachment is attachable (and/or attached) to the hair dryer for ergonomic use of one or both of the blower and the aerosol dye applicator, such that a user does not need to change their grip or change out a device being held for use of these features of the combination.

In embodiments, a spray area of the aerosol attachment and, optionally, a portion of the hot air blown by the hair dryer, can be at least partially coincident, as a non-limiting example. This can facilitate a user to both style and color the hair with a combination device. As such, in embodiments, the cosmetic composition can be applied with a use of a combination device that includes the attachment and the hair dryer. In embodiments, application of the cosmetic composition can be adjusted, for example with a use of the combination device, for coating and coverage of the hair with the cosmetic composition. Example adjustments include activation and/or deactivation of application of the cosmetic composition as needed, adjustment of a distance between the combination device and the hair as needed, activation and/or deactivation of the aerosol attachment and/or the blower of the hair dryer as needed, and the like.

In embodiments, the hair can be treated with shampoo, conditioner, plasma, hot air, or any combination thereof, to change the surface texture of the hair and enhance penetration of the cosmetic composition into and adhesion to the hair. In embodiments, the hair is treated with shampoo before, during, and/or after application of the cosmetic composition to the hair. In embodiments, the hair is treated with conditioner before, during, and/or after application of the cosmetic composition to the hair. In embodiments, the hair is treated with plasma before, during, and/or after application of the cosmetic composition to the hair. In embodiments, the hair is treated with warm and/or hot air before, during, and/or after application of the cosmetic composition to the hair. In embodiments, an ion generator can be used to activate the aerosolized cosmetic composition, e.g., prior to application to the hair. In embodiments, one or more of these and/or other treatment(s) can be applied with a use of the combination device, for example.

In embodiments, the intensity of the aerosol attachment and/or the hair dryer blower, such as rate of aerosol and/or air movement and/or level of heat, can be adjusted as part of the application process. Such adjustments can be made based on a distance of the device from the hair or scalp of an individual, for example, to avoid excessive heating or damage to the hair or scalp. In embodiments, the cosmetic composition can be aerosolized and/or provided as a consumable for a use with the combination device; for example, the cosmetic composition can be provided as a cartridge or the like.

Referring back to FIG. 2, in at least some embodiments, an aerosol attachment device 2 can be configured for emission of an infrared (IR) light beam. In embodiments, the IR light beam can comprise, consist essentially of, or consist of a near IR band. In embodiments, the near IR band can be within the range of 650 to 850 nm wavelength, or any portion thereof. The IR light beam can, in embodiments, cure a cosmetic composition that is configured for management or improvement of hair fibers, for example. In embodiments, a cosmetic composition that is curable with the IR light beam is not or does not include a hair coloration composition.

In embodiments, a combination device includes a thermal sensor, placed within a middle portion of an air stream of the hair dryer, where the blown air is warmest. The thermal sensor can be configured to measure temperature of the blown air. The thermal sensor can be operably connected to control circuitry of the combination device; in embodiments, the control circuitry can increase or decrease temperature of the blown air by controlled activation or deactivation of a heating element of the hair dryer, for example. In embodiments, the control circuitry can be configured for computation of a cumulative power projected onto or near the individual's hair or scalp, as a sum of power output that includes the hair dryer. In embodiments, temperature of the blown air can be prioritized for reduction in the event that the cumulative power is above a threshold value, such that a hair coloration process (e.g., with a hair color composition) is not negatively impacted by excessive drying of the hair. In embodiments, the hot air blower can be automatically decreased or deactivated if the cumulative power is above a threshold value, for example, to reduce the risk of injury to the hair or scalp.

In various embodiments, the combination device can be a “smart hair dryer” that includes, among other features, one or more sensors (e.g., thermal sensor) that are operably connected with circuitry configured for performing logic operations based on data received from the one or more sensors for automated, semi-automated, or assisted manual application of the cosmetic composition to a portion of the body of an individual. The logic operations can include all or part of a method and/or process of the disclosure, for example, optionally in combination with additional logic operations. Attachment devices, hair dryers, and/or devices or systems of the disclosure can be provided as kits that include instructional materials configured to direct a user in a use of the kit.

As such, in aspects, the disclosure provides a “smart hair dryer” computational system or device comprising circuitry configured to perform all or part of a method, and in at least some aspects, a system and/or network of computational devices comprising at least part of such circuitry. In embodiments, a smart hair dryer computational device can include a form factor that corresponds to a form factor of a hair dryer, a hair dryer system, or the like, as disclosed herein. In embodiments, circuitry of such a computational device is configurable with a processor and processor-executable instructions stored on a non-transitory machine-readable medium of the computational device, as a non-limiting example, but other approaches for configuring circuitry of the computational device can be implemented in embodiments. In embodiments, the computational device includes a processor for execution of instructions stored on a non-transitory machine-readable medium, for enabling the processor to carry out all or part of a method or process of the disclosure. Accordingly, in embodiments, a computational device includes a software application configured to perform all or part of one or more methods or processes of the disclosure, in any order or combination. However, in embodiments, a computational device includes dedicated hardware circuitry. Further configuration of circuitry of the computational device can include networking circuitry, for example, circuitry configured for a wireless connection, such as a Bluetooth® connection, a Bluetooth® low energy (BLE) connection, and/or a Wi-Fi® connection, and/or a wired connection. The networking circuitry, in combination with circuitry of the computational device, can be used to request, retrieve, and/or receive data from a remote server, for example. In embodiments, the circuitry can include operable connection of one or more sensors with the processor, or other circuitry, for performing logic operations and/or methods based on data received from the one or more sensors, for example.

### Terminology

As used herein, including in the claims, the terms “a,” “an,” “the,” and the like, refer to the singular and the plural forms of the object or element referenced. The term “comprising” is inclusive or open-ended and does not exclude additional, unrecited elements or steps. The term “consisting of,” as used in a claim, excludes any element, step, or ingredient not specified in the claim. The term “consisting essentially of,” as used in a claim, limits the scope of the claim to the specified materials or steps and those that do not materially affect the basic and novel characteristic(s) of the claim.

As used herein, the term “device,” and similar terms, refers to an electronic device that is configured, or that can be configured, to perform all or part of a method or process of the disclosure. The term “system,” and similar terms, refers to two or more, i.e., multiple, devices that together are configured, or can be configured, to perform all or part of a method or process of the disclosure. Devices and systems can be operably connected by one or more electronic telecommunications connections or network connections for electronic communications that utilize one or more networking protocols for the transmission or exchange of data, for example, for carrying out all or part of a method as a computational method. The term “computational method” refers to a method, or a portion of a method, that is carried out substantially or entirely by a device or system, with little or no human or manual input.

As used herein, the term “diagnosis,” “analysis,” and similar terms, refer to one or more variable values, computed by circuitry from imagery of hair of an individual captured by a camera sensor, that correspond to, or are descriptive of, the status of the hair of the individual. A diagnosis can be comprised of or computed from measurements of observable characteristics of the hair, such as strand thickness, density, length, color, type, degree of wetness, degree of dryness, degree of curing of a cosmetic composition, degree of coverage of the hair with the cosmetic composition, and the like.

As used herein, the term “applicator spray level” refers to a level of spraying of an applicator, and can include variations in spray rate, spray area, spray focal area, spray aperture, or other variables that relate to operation of the applicator.

As used herein, the term “circuitry” refers to electronic and/or telecommunications circuitry configured to perform one or more logic operations and/or methods of the disclosure, in whole or in part, in any order or combination. Non-limiting examples of circuitry include a non-transitory machine-readable storage medium having stored thereon instructions which, when executed by a processor (e.g., microprocessor), configure or cause the processor to perform all or part of a method or computational method of the disclosure. Other non-limiting examples of circuitry include dedicated computational hardware circuitry configured for logic operations.

As used herein, the term “instructional material,” and similar terms, refers to any material or media, whether print, audio, visual, illustrative, audiovisual, tactile, or otherwise, that can convey to an individual information about how to use a kit, device, system, or other aspect of the disclosure in a method or process for remote sebum measurement.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. An attachment for a hair dryer, the attachment comprising a camera sensor operably connected to circuitry; wherein the circuitry is configured to image, with the camera sensor, a portion of a body of an individual for a diagnosis of the portion of the body and an application of an aerosolized cosmetic composition to the portion of the body based at least in part on the diagnosis.

Embodiment 2. The attachment of Embodiment 1 or any other Embodiment, wherein the attachment is configured for physical attachment to the hair dryer.

Embodiment 3. The attachment of any one of Embodiments 1-2 or any other Embodiment, wherein the attachment is an after-market addition to the hair dryer.

Embodiment 4. The attachment of any one of Embodiments 1-3 or any other Embodiment, wherein the aerosolized cosmetic composition is for application to hair of an individual.

Embodiment 5. The attachment of any one of Embodiments 1-4 or any other Embodiment, wherein the aerosolized cosmetic composition includes a hair dye that is curable with hot air of the hair dryer, wherein the attachment includes an ion generator for generation of ions in the hair dye prior to application to hair of the individual.

Embodiment 6. The attachment of any one of Embodiments 1-6 or any other Embodiment, wherein the diagnosis corresponds to a natural strand color, a strand density, a strand thickness, a strand length, one or more artificial strand colors, or any combination thereof.

Embodiment 7. The attachment of any one of Embodiment 1-6 or any other Embodiment, wherein the circuitry is configured to compute a hair dryer blower level, a hair dryer blower temperature, and/or an applicator spray level based at least in part on the diagnosis.

Embodiment 8. The attachment of any one of Embodiments 1-7 or any other Embodiment, wherein the circuitry is configured to: activate an applicator of the attachment for application of an aerosolized cosmetic composition to the portion of the body of the individual; and activate a blower of the hair dryer; wherein the hair dryer blows air based at least in part on the hair dryer blower level, the hair dryer blower temperature, and/or the applicator spray level.

Embodiment 9. A hair dryer system comprising the attachment of any one of Embodiments 1-8 or any other Embodiment, wherein the attachment is attached or is attachable a hair dryer of the hair dryer system.

Embodiment 10. A method for applying an aerosolized cosmetic composition to a portion of a body of an individual, the method comprising: imaging, with a camera sensor of an attachment for a hair dryer, the portion of the body to produce an image, wherein the attachment is attachable to the hair dryer; computing, with circuitry, a diagnosis of the portion of the body of the individual based at least in part on the image; computing, with circuitry, a hair dryer blower level, a hair dryer blower temperature, and/or an applicator spray level based at least in part on the diagnosis; applying, with an applicator, the aerosolized cosmetic composition to the portion of the body of the individual, and activating a blower of the hair dryer; wherein the hair dryer blows air based at least in part on the hair dryer blower level, the hair dryer blower temperature, and/or the applicator spray level.

Embodiment 11. The method of Embodiment 10 or any other Embodiment, wherein the attachment is attached to the hair dryer.

Embodiment 12. The method of any one of Embodiments 10-11 or any other Embodiment, wherein a diagnosis area of the camera sensor, a spray of the applicator of the attachment, and a portion of the hot air blown by the hair dryer are coincident with the portion of the body.

Embodiment 13. The method of any one of Embodiments 10-12 or any other Embodiment, wherein the aerosolized cosmetic composition is for application to hair of the individual.

Embodiment 14. The method of any one of Embodiments 10-13 or any other Embodiment, wherein the aerosolized cosmetic composition includes a hair dye that is curable with hot air of the hair dryer.

Embodiment 15. A computational device comprising circuitry configured to perform all or part of the method of any one of Embodiments 10-14 or any other Embodiment.

While illustrative embodiments have been illustrated and described, it will be appreciated that various changes can be made therein without departing from the spirit and scope of the disclosure.

The embodiments of the invention in which an exclusive property or privilege is claimed are defined as follows:

1. An attachment for a hair dryer, the attachment comprising a camera sensor operably connected to circuitry;
wherein the circuitry is configured to image, with the camera sensor, a portion of a body of an individual for a diagnosis of the portion of the body and an application of an aerosolized cosmetic composition to the portion of the body based at least in part on the diagnosis.

2. The attachment of claim 1, wherein the attachment is configured for physical attachment to the hair dryer.

3. The attachment of claim 2, wherein the attachment is an after-market addition to the hair dryer.

4. The attachment of claim 1, wherein the aerosolized cosmetic composition is for application to hair of an individual.

5. The attachment of claim 4, wherein the aerosolized cosmetic composition includes a hair dye that is curable with hot air of the hair dryer, wherein the attachment includes an ion generator for generation of ions in the hair dye prior to application to hair of the individual.

6. The attachment of claim 1, wherein the diagnosis corresponds to a natural strand color, a strand density, a strand thickness, a strand length, one or more artificial strand colors, or any combination thereof.

7. The attachment of claim 1, wherein the circuitry is configured to compute a hair dryer blower level, a hair dryer blower temperature, and/or an applicator spray level based at least in part on the diagnosis.

8. The attachment of claim 7, wherein the circuitry is configured to:
activate an applicator of the attachment for application of an aerosolized cosmetic composition to the portion of the body of the individual; and
activate a blower of the hair dryer; wherein the hair dryer blows air based at least in part on the hair dryer blower level, the hair dryer blower temperature, and/or the applicator spray level.

9. A hair dryer system comprising the attachment of claim 1, wherein the attachment is attached or is attachable a hair dryer of the hair dryer system.

10. A method for applying an aerosolized cosmetic composition to a portion of a body of an individual, the method comprising:
imaging, with a camera sensor of an attachment for a hair dryer, the portion of the body to produce an image, wherein the attachment is attachable to the hair dryer;
computing, with circuitry, a diagnosis of the portion of the body of the individual based at least in part on the image;
computing, with circuitry, a hair dryer blower level, a hair dryer blower temperature, and/or an applicator spray level based at least in part on the diagnosis;
applying, with an applicator, the aerosolized cosmetic composition to the portion of the body of the individual, and activating a blower of the hair dryer; wherein the hair dryer blows air based at least in part on the hair dryer blower level, the hair dryer blower temperature, and/or the applicator spray level.

11. The method of claim 10, wherein the attachment is attached to the hair dryer.

12. The method of claim 11, wherein a diagnosis area of the camera sensor, a spray of the applicator of the attachment, and a portion of the hot air blown by the hair dryer are coincident with the portion of the body.

13. The method of claim 10, wherein the aerosolized cosmetic composition is for application to hair of the individual.

14. The method of claim 13, wherein the aerosolized cosmetic composition includes a hair dye that is curable with hot air of the hair dryer.

15. A computational device comprising circuitry configured to perform all or part of the method of claim 10.
